(19) United States
(12) Patent Application Publication
(10) Pub. No.: US 2015/0005269 A1
(43) Pub. Date: Jan. 1, 2015

(54) Treatment of Heart Failure and Sudden Cardiac Death

(71) Applicant: Rhode Island Hospital, Providence, RI (US)

(72) Inventors: Gideon Koren, Brookline, MA (US); Zhe Zheng, Riverside, RI (US)

(21) Appl. No.: 14/370,126

(22) PCT Filed: Jan. 3, 2013
(86) PCT No.: PCT/US13/20102

§ 371 Date: Jul. 1, 2014

Related U.S. Application Data
(60) Provisional application No. 61/582,583, filed on Jan. 3, 2012.

(51) Int. Cl. A61K 31/575; A61K 45/06

(52) U.S. Cl. CPC A61K 31/575; A61K 45/06
USPC 514/171; 552/554; 514/179

(57) ABSTRACT

A method of reducing heart failure or preventing sudden cardiac death in a subject in need thereof is carried out by administering a composition comprising a compound that increases cellular level of Sarco/endoplasmic reticulum Ca²⁺-ATPase 2a (SERCA2a). The invention represents a significant breakthrough in the treatment or prevention of chronic heart failure. The invention is based upon the discovery that a Mifepristone molecule increases the cellular level of SERCA2a protein.

### RELATED APPLICATION

This application claims the benefit and priority to U.S. Ser. No. 61/582,583, filed Jan. 3, 2012, the contents of which are incorporated herein by reference in their entireties.

### GOVERNMENT SUPPORT

This invention was made with Government support under RO1 HL093205-01A1 and T32 HL094300 awarded by the National Institutes of Health. The Government has certain rights in the invention.

### FIELD OF THE INVENTION

The invention relates to treatment of cardiac pathologies.

### BACKGROUND OF THE INVENTION

Chronic heart failure is a common end stage of cardiovascular disease and there are annually 50,000 new diagnosed patients in the United States with the average mortality rate of 10%-35%. There is a strong need for novel and effective therapeutic approach to treat or prevent this disease.

### SUMMARY OF THE INVENTION

The invention represents a significant breakthrough in the treatment or prevention of chronic heart failure. The invention is based upon the discovery that a mifepristone molecule increases the cellular level of Sarco/endoplasmic reticulum Ca²⁺-ATPase 2a (SERCA2a) protein.

Accordingly, the present invention provides a composition for reducing heart failure in a subject in need of, where the composition comprises a compound that increases cellular level of Sarco/endoplasmic reticulum Ca²⁺-ATPase 2a (SERCA2a). The composition may comprise mifepristone or a derivative. The composition may also comprise a progesterone receptor modulator that is an antagonist or partial agonist. A method of reducing heart failure or preventing sudden cardiac death in a subject in need thereof is carried out by administering a composition comprising a compound that increases cellular level of SERCA2a such as a progesterone receptor modulator (e.g., an antagonist or a partial agonist). In preferred embodiments, the progesterone receptor modulator comprises a progesterone receptor antagonist such as mifepristone or a partial agonist of the progesterone receptor. Preferably, the compound is a small molecule.

A “small molecule” as used herein, is meant to refer to a composition that has a molecular weight of less than about 2500 daltons. A small molecule is generally a compound that is less than 2000 daltons in mass. The molecular mass of the small molecule is preferably less than 1000 daltons, more preferably less than 600 daltons, e.g., the compound is less than 500 daltons, 400 daltons, 300 daltons, 200 daltons, or 100 daltons. Small molecules can be, e.g., nucleic acids, peptides, polypeptides, peptidomimetics, carbohydrates, lipids or other organic or inorganic molecules. In preferred embodiments, the upper molecular weight limit for a small molecule is approximately 800 daltons, which permits the molecule to rapidly diffuse across cell membranes so that it reaches intracellular sites of action.

The composition comprises mifepristone or a derivative thereof. In some embodiments, the composition further comprises a second, third, or fourth therapeutically active compound. For example, the composition comprises a potassium sparing diuretic such as spironolactone or eplerenone.

Accordingly, the invention provides methods of reducing heart failure or methods of preventing sudden cardiac death in a subject in need thereof by administering a composition comprising mifepristone or a derivative thereof at a dose that increases cellular level of SERCA2a. A subject in need of such therapeutic intervention is characterized by one or more symptoms of heart failure. For example, the subject is diagnosed with class 3 or class 4 heart failure and/or diastolic heart failure. Heart failure may be a consequence of cardiomyopathy, coronary artery disease, chronic hypertension, or other cardiovascular disorders. A typical subject or candidate for treatment has not been diagnosed with Cushing's Disease. Treatment using the compositions and methods described herein leads to a clinically relevant improvement in cardiac function. In preferred embodiments, the subject has not been diagnosed as pregnant.

Also provided is a method of preventing or inhibiting a cardiovascular disease in a subject in need thereof by assessing the level of cardiac impairment or determining the level of Sarco/endoplasmic reticulum Ca2+-ATPase 2a (SERCA2a) in the subject; administering to the subject a therapeutically effective amount of mifepristone, in an amount effective to increase the level of SERCA2a; and re-assessing the level of cardiac impairment and/or the level of SERCA2a in said subject. A reduction in the level of cardiac impairment and/or an increase in the level of SERCA2a after administration of mifepristone is indicative of clinical benefit, e.g., reduced risk of having a cardiovascular disease or impairment in the subject.

The compound of the present invention can be administered prior to, concurrently, or after administering a second composition comprising a non-nucleic acid based composition. The level of cardiac impairment in a subject comprises class III or class IV heart failure.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

Compounds used in the invention are purified. Polynucleotides, polypeptides, or other agents are purified and/or isolated. For example, a compound has been isolated or purified from chemical precursors or other chemicals when chemically synthesized. Purified compounds are at least 60% by weight (dry weight) the compound of interest. Preferably, the preparation is at least 75%, more preferably at least 90%, and most preferably at least 99%, by weight the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis. Purified also defines a degree of sterility that is safe for administration to a human subject, e.g., lacking infectious or toxic agents.

Treatment of heart failure with a small molecule delivered as a pill that increases SERCA2a level in the heart increases the efficiency of therapy, lowers the risk and the cost of treatment. Another advantage of treatment using a small molecule is that such therapy is available to patients in an outpatient setting. In contrast, gene therapy with SERCA2a necessitates admission to a hospital, invasive procedure such as cardiac catheterization and gene delivery to the heart tissue, which can induce inflammation. Thus, this procedure can only be done in tertiary care facility for selected individuals.

Other features and advantages of the invention will be apparent from the following description of the preferred embodiments thereof, and from the claims. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All published foreign patents and patent applications cited herein are incorporated herein by reference.

### BRIEF DESCRIPTION OF THE DRAWINGS

FIG. 1 is a series of fluorescence of images of neonatal rabbit ventricular cardiomyocytes (NRbVCM). A. Cardiomyocytes were immunostained with anti-Sarcomeric-α-actinin (red) antibody and detected with Alexa 594 donkey anti-mouse IgG. B. Fibroblasts were immunostained with anti-Vemintin (green) antibody and detected with Alexa 488 goat anti-rat IgG. All nuclei were stained with DAPI.

FIG. 2A is a photograph of a Western blot of cell lysates of NRbVCM on 2011-04-04. FIG. 2B is a photograph of a Western blot of cell lysates of NRbVCM.

FIG. 3 is a bar graphs showing the results of a Western blot assay. NRbVCMs were treated with vehicle, 1 nM of progesterone, and 50 nM of RU486, respectively, and harvested after 48 hours. Protein level of SERCA2 was analyzed by western blots. All measurements were normalized against GAPDH. Bar graphs represent the averages of three independent experiments plus S.D. in arbitrary unit. * p<0.05.

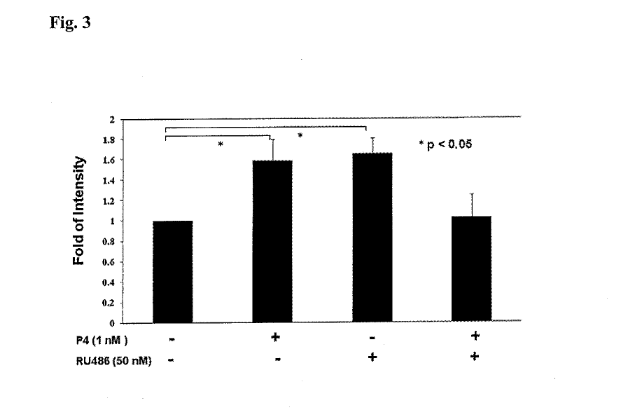

### DETAILED DESCRIPTION

Heart failure is induced by different etiologies such as coronary artery disease, hypertension, diabetes, infection, or inflammation and generally results in calcium cycling dysregulation at the myocyte level. Cardiac gene therapy using SERCA2a has been used for the treatment of heart failure (del Monte et al., 1999, Circulation 100:2308-2311; Gwathmey et al., 2011, Journal of Molecular and Cellular Cardiology 50: 803-812). However, gene therapy approaches can be associated with drawbacks as described above.

Mifepristone or derivative thereof was found to increase the cellular level of SERCA2a protein in cardiac myocytes. For example, cardiac cells are contacted with the molecule and the molecule slows the degradation of SERCA2a protein in the cell, thereby leading to an increase in the protein level of this gene product. Accordingly, the invention provides methods of reducing heart failure or preventing sudden cardiac death by administering a composition comprising mifepristone or a derivative thereof at a dose that increases cellular level of SERCA2a or other progesterone receptor modulators, e.g., antagonists or partial agonists, that increase the level of SERCA2a in a cell.

### Mifepristone and its Derivatives

RU486 (Mifepristone) is the well-known progesterone receptor antagonist that has been widely used in abortion and postcoital contraception. Mifepristone is also known by the following designations or names: RU486; RU38486; ZK 98296; RU 486; RU 38486; Mifepristone Exelgyn Brand; Mifepristone Danco Brand; R 38486; Mifepristone Contragest Brand; ZK98296; ZK-98296; RU-486; RU-38486; R38486; R-38486; Mif+-gyne; Mifeprex; Mifegyne; Exelgyn Brand of Mifepristone; or Danco Brand of Mifepristone and is described in patents such as U.S. Pat. No. 4,386,085 (A), U.S. Pat. No. 4,447,424 (A), U.S. Pat. No. 4,519,946 (A), U.S. Pat. No. 4,547,493 (A), U.S. Pat. No. 4,634,695 (A), U.S. Pat. No. 4,634,696 (A), U.S. Pat. No. 4,978,657 (A), U.S. Pat. No. 5,006,518 (A), and U.S. Pat. No. 5,043,332 (A), hereby incorporated by reference.

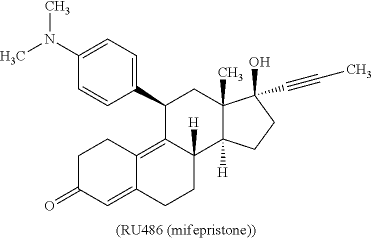

In one embodiment, the mifepristone derivative is a compound of the formula:

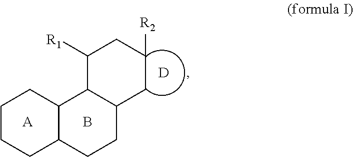

or a pharmaceutically acceptable salt, solvate, or prodrug thereof, wherein A and B are rings selected from:

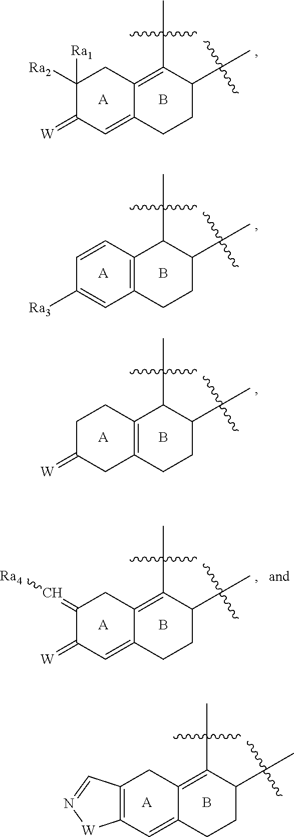

### W is O, S, or NH;

Ra₁ and Ra₂ are independently, H, CN, or C₁-C₄ alkyl;
Ra₃ is H, OH, O(C₁-C₄ alkyl), or C(O)(C₁-C₄ alkyl);
Ra₄ is NH₂, NH(C₁-C₄ alkyl), N(C₁-C₄ alkyl)₂, or N(Ra₅)₂ where two Ra₅ taken together with the nitrogen atom form a 5-6 membered heterocycle optionally containing 1-3 heteroatoms selected from N, O and S; Ra₄ may be in the Z or E position;
R₁ is optionally unsaturated C₁-C₁₂ alkyl, aryl, heteroaryl, or aralkyl, wherein alkyl, aryl, heteroaryl, and aralkyl may be substituted;
R₂ is unsubstituted or substituted C₁-C₄ alkyl; and
D is 5-6 membered carbocycle, 5-6 membered heterocycle, 5-6 membered aryl, or 5-6 membered heteroaryl, wherein the carbocycle, heteroaryl, aryl and heteroaryl may be substituted.

In one embodiment, the mifepristone derivative is a compound of the formula:

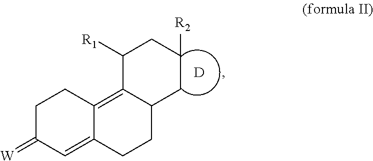

where R₁, R₂, W, and D are as defined above, or a pharmaceutically acceptable salt, solvate, or prodrug thereof.

In one embodiment, the mifepristone derivative is a compound of the formula:

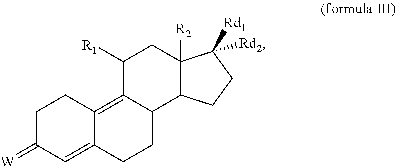

where R₁, R₂ and W are as defined above, wherein Rd₁ is H, OH, O(C₁-C₄ alkyl), or C(O)(C₁-C₄ alkyl); and
Rd₂ is H, C₁-C₄ alkyl, C₂-C₄ alkenyl, or C₂-C₄ alkynyl,
or a pharmaceutically acceptable salt, solvate, or prodrug thereof.

In one embodiment, the mifepristone derivative is a compound of the formula:

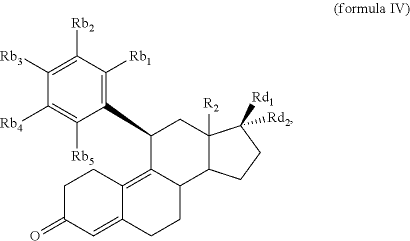

wherein R₂, Rd₁, and Rd₂ are as defined above,
Wherein Rb₁, Rb₂, Rb₃, Rb₄ and Rb₅ are independently, H, C₁-C₄ alkyl, OH, O(C₁-C₄ alkyl), NH₂, NH(C₁-C₄ alkyl), or N(C₁-C₄ alkyl)₂,
or a pharmaceutically acceptable salt, solvate, or prodrug thereof.

In one embodiment, the mifepristone derivative is a compound of the formula:

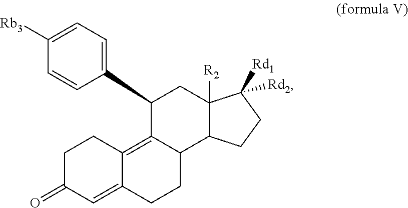

wherein R₂, Rd₁, Rd₂ and Rb₃ are as defined above, or a pharmaceutically acceptable salt, solvate, or prodrug thereof.

Mifepristone derivatives have been described, e.g., mifepristone with different linker groups in position 4′ of the phenyl ring in the 11β position of the steroid scaffold (Ho et al., 2009, J. Med. Chem. 52: 1268-1274; hereby incorporated by reference). Other derivatives include ZK 112993 (CAS 105114-63-4; Anticancer Res 1990; 10(3):683); RU 40555 (CAS 145380-08-1; J Rheumatol 1992 February; 19(2):216-22); RU 42633 (CAS 104004-96-8; Prog Clin Biol Res 1989; 300:445); RU49953 (J Neurochem 2001 February; 76(4):1121-8); mifepristone methochloride (CAS 109345-60-0; Ophthalmic Res 1987; 19(1):61); RU 45196 (CAS 121548-81-0; J Steroid Biochem; 32(3):347); ZK 114043 (CAS 134235-42-0; J Cell Physiol 1995 July; 164(1):1-8); RU 46534 (J Reprod Fertil Suppl 1997; 51:317-25); RU 42698 (CAS 105012-15-5; Contraception 1993 August; 48(2):133-49); RU 42848 (CAS 104004-92-4; Contraception 1993 August; 48(2):133-49); 4′-(dimethylamino)-17-hydroxy-17-(1-propynyl)benzo(12,12a)-11,18-cyclo-12a,12b-dihomo-estr-4-en-3-one (CAS 156383-09-4; Steroids 1994 March; 59(3):185-90: 11-(4-dimethylaminophenyl)-17-hydroxy-17-(3-methyl-1-butynyl)-4,9-estradien-3-one (Steroids 2000 March; 65(3):157-62); and 11-(4-acetophenyl)-17-hydroxy-17-(3-methyl-1-butynyl)-4,9-estradien-3-one (Steroids 2000 March; 65(3):157-62). Each of the references listed above describing RU486 derivatives is hereby incorporated by reference.

“Pharmaceutically acceptable salt” of a compound means a salt that is pharmaceutically acceptable and that possesses the desired pharmacological activity of the parent compound.

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicyclic, stearic, subacetic, succinic, sulfamic, sulfanilic, sulfuric, tannic, tartaric, toluene sulfonic, and the commonly occurring amine acids, e.g., glycine, alanine, phenylalanine, arginine, etc.

“Solvates” means solvent addition forms that contain either stoichiometric or non stoichiometric amounts of solvent. Some compounds have a tendency to trap a fixed molar ratio of solvent molecules in the crystalline solid state, thus forming a solvate. If the solvent is water the solvate formed is a hydrate, when the solvent is alcohol, the solvate formed is an alcoholate. Hydrates are formed by the combination of one or more molecules of water with one of the substances in which the water retains its molecular state as H₂O, such combination being able to form one or more hydrate.

The terms “pro-drug” and “prodrug” are used interchangeably herein and refer to any compound which releases an active parent drug in vivo. Since prodrugs are known to enhance numerous desirable qualities of pharmaceuticals (e.g., solubility, bioavailability, manufacturing, etc.) mifepristone or its derivative can be delivered in prodrug form. Thus, the present invention is intended to cover prodrugs of mifepristone or its derivative, methods of delivering the same and compositions containing the same. “Prodrugs” are intended to include any covalently bonded carriers that release an active parent drug of the present invention in vivo when such prodrug is administered to a subject. Prodrugs the present invention are prepared by modifying functional groups present in the compound in such a way that the modifications are cleaved, either in routine manipulation or in vivo, to the parent compound. Prodrugs include mifepristone or its derivative wherein a hydroxy, amino, sulfhydryl, carboxy, or carbonyl group is bonded to any group that, may be cleaved in vivo to form a free hydroxyl, free amino, free sulfhydryl, free carboxy or free carbonyl group, respectively.

The term “substituted,” as used herein, means that any one or more hydrogens on the designated atom is replaced with a selection from the indicated group, provided that the designated atom's normal valency is not exceeded, and that the substitution results in a stable compound. When a substituent is keto (i.e., ═O), then 2 hydrogens on the atom are replaced. Keto substituents are not present on aromatic moieties. Ring double bonds, as used herein, are double bonds that are formed between two adjacent ring atoms (e.g., C═C, C═N, or N═N).

As used herein, “alkyl” is intended to include both branched and straight-chain saturated aliphatic hydrocarbon groups having the specified number of carbon atoms. For example, C₁₋₆ alkyl is intended to include C₁, C₂, C₃, C₄, C₅, and C₆ alkyl groups. Examples of alkyl include, but are not limited to, methyl, ethyl, n-propyl, i-propyl, n-butyl, s-butyl, t-butyl, n-pentyl, s-pentyl, and n-hexyl. “Alkyl” further includes alkyl groups that have oxygen, nitrogen, sulfur or phosphorous atoms replacing one or more hydrocarbon backbone carbon atoms. In certain embodiments, a straight chain or branched chain alkyl has six or fewer carbon atoms in its backbone (e.g., C₁-C₆ for straight chain, C₃-C₆ for branched chain), and more preferably four or fewer. Likewise, preferred cycloalkyls have from three to eight carbon atoms in their ring structure, and more preferably have five or six carbons in the ring structure.

The term “alkyl” also includes both “unsubstituted alkyls” and “substituted alkyls”, the latter of which refers to alkyl moieties having substituents replacing a hydrogen on one or more carbons of the hydrocarbon backbone. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Cycloalkyls can be further substituted, e.g., with the substituents described above. An “alkylaryl” or an “aralkyl” moiety is an alkyl substituted with an aryl (e.g., phenylmethyl (benzyl)).

“Alkenyl” includes unsaturated aliphatic groups analogous in length and possible substitution to the alkyls described above, but that contain at least one double bond. For example, the term “alkenyl” includes straight-chain alkenyl groups (e.g., ethenyl, propenyl, butenyl, pentenyl, hexenyl, heptenyl, octenyl, nonenyl, decenyl), branched-chain alkenyl groups, cycloalkenyl (e.g., alicyclic) groups (e.g., cyclopropenyl, cyclopentenyl, cyclohexenyl, cycloheptenyl, cyclooctenyl), alkyl or alkenyl substituted cycloalkenyl groups, and cycloalkyl or cycloalkenyl substituted alkenyl groups. The term “alkenyl” further includes alkenyl groups, which include oxygen, nitrogen, sulfur or phosphorous atoms replacing one or more hydrocarbon backbone carbons. In certain embodiments, a straight chain or branched chain alkenyl group has six or fewer carbon atoms in its backbone (e.g., C₂-C₆ for straight chain, C₃-C₆ for branched chain). Likewise, cycloalkenyl groups may have from three to eight carbon atoms in their ring structure, and more preferably have five or six carbons in the ring structure. The term “C₂-C₆” includes alkenyl groups containing two to six carbon atoms. The term “C₃-C₆” includes alkenyl groups containing three to six carbon atoms.

The term “alkenyl” also includes both “unsubstituted alkenyls” and “substituted alkenyls”, the latter of which refers to alkenyl moieties having substituents replacing a hydrogen on one or more hydrocarbon backbone carbon atoms. Such substituents can include, for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

“Alkynyl” includes unsaturated aliphatic groups analogous in length and possible substitution to the alkyls described above, but which contain at least one triple bond. For example, “alkynyl” includes straight-chain alkynyl groups (e.g., ethynyl, propynyl, butynyl, pentynyl, hexynyl, heptynyl, octynyl, nonynyl, decynyl), branched-chain alkynyl groups, and cycloalkyl or cycloalkenyl substituted alkynyl groups. The term “alkynyl” further includes alkynyl groups having oxygen, nitrogen, sulfur or phosphorous atoms replacing one or more hydrocarbon backbone carbons. In certain embodiments, a straight chain or branched chain alkynyl group has six or fewer carbon atoms in its backbone (e.g., C₂-C₆ for straight chain, C₃-C₆ for branched chain). The term “C₂-C₆” includes alkynyl groups containing two to six carbon atoms. The term “C₃-C₆” includes alkynyl groups containing three to six carbon atoms.

The term “alkynyl” also includes both “unsubstituted alkynyls” and “substituted alkynyls”, the latter of which refers to alkynyl moieties having substituents replacing a hydrogen on one or more hydrocarbon backbone carbon atoms. Such substituents can include, for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

“Aryl” includes groups with aromaticity, including 5- and 6-membered “unconjugated”, or single-ring, aromatic groups that may include from zero to four heteroatoms, as well as “conjugated”, or multicyclic, systems with at least one aromatic ring. Examples of aryl groups include benzene, phenyl, pyrrole, furan, thiophene, thiazole, isothiazole, imidazole, triazole, tetrazole, pyrazole, oxazole, isooxazole, pyridine, pyrazine, pyridazine, and pyrimidine, and the like. Furthermore, the term “aryl” includes multicyclic aryl groups, e.g., tricyclic, bicyclic, e.g., naphthalene, benzoxazole, benzodioxazole, benzothiazole, benzoimidazole, benzothiophene, methylenedioxyphenyl, quinoline, isoquinoline, napthridine, indole, benzofuran, purine, benzofuran, deazapurine, or indolizine. Those aryl groups having heteroatoms in the ring structure may also be referred to as “aryl heterocycles”, “heterocycles,” “heteroaryls” or “heteroaromatics”. The aromatic ring can be substituted at one or more ring positions with such substituents as described above, as for example, halogen, hydroxyl, alkoxy, alkylcarbonyloxy, arylcarbonyloxy, alkoxycarbonyloxy, aryloxycarbonyloxy, carboxylate, alkylcarbonyl, alkylaminocarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, cyano, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Aryl groups can also be fused or bridged with alicyclic or heterocyclic rings, which are not aromatic so as to form a multicyclic system (e.g., tetralin, methylenedioxyphenyl).

As used herein, “carbocycle” or “carbocyclic ring” is intended to mean any stable monocyclic, bicyclic, or tricyclic ring having the specified number of carbons, any of which may be saturated, unsaturated, or aromatic. For example a C₃₋₁₄ carbocycle is intended to mean a mono-, bi-, or tricyclic ring having 3, 4, 5, 6, 7, 8, 9, 10, 11, 12, 13, or 14 carbon atoms. Examples of carbocycles include, but are not limited to, cyclopropyl, cyclobutyl, cyclobutenyl, cyclopentyl, cyclopentenyl, cyclohexyl, cycloheptenyl, cycloheptyl, cycloheptenyl, adamantyl, cyclooctyl, cyclooctenyl, cyclooctadienyl, fluorenyl, phenyl, naphthyl, indanyl, adamantyl, and tetrahydronaphthyl. Bridged rings are also included in the definition of carbocycle, including, for example, [3.3.0]bicyclooctane, [4.3.0]bicyclononane, [4.4.0]bicyclodecane, and [2.2.2]bicyclooctane. A bridged ring occurs when one or more carbon atoms link two non-adjacent carbon atoms. Preferred bridges are one or two carbon atoms. It is noted that a bridge always converts a monocyclic ring into a tricyclic ring. When a ring is bridged, the substituents recited for the ring may also be present on the bridge. Fused (e.g., naphthyl and tetrahydronaphthyl) and spiro rings are also included.

As used herein, the term “heterocycle” or “heterocyclic” is intended to mean any stable monocyclic, bicyclic, or tricyclic ring which is saturated, unsaturated, or aromatic and comprises carbon atoms and one or more ring heteroatoms, e.g., 1 or 1-2 or 1-3 or 1-4 or 1-5 or 1-6 heteroatoms, independently selected from the group consisting of nitrogen, oxygen, and sulfur. A bicyclic or tricyclic heterocycle may have one or more heteroatoms located in one ring, or the heteroatoms may be located in more than one ring. The nitrogen and sulfur heteroatoms may optionally be oxidized (i.e., N→O and S(O)ₚ, where p=1 or 2). When a nitrogen atom is included in the ring it is either N or NH, depending on whether or not it is attached to a double bond in the ring (i.e., a hydrogen is present if needed to maintain the tri-valency of the nitrogen atom). The nitrogen atom may be substituted or unsubstituted (i.e., N or NR wherein R is H or another substituent, as defined). The heterocyclic ring may be attached to its pendant group at any heteroatom or carbon atom that results in a stable structure. The heterocyclic rings described herein may be substituted on carbon or on a nitrogen atom if the resulting compound is stable. A nitrogen in the heterocycle may optionally be quaternized. It is preferred that when the total number of S and O atoms in the heterocycle exceeds 1, then these heteroatoms are not adjacent to one another. Bridged rings are also included in the definition of heterocycle. A bridged ring occurs when one or more atoms (i.e., C, O, N, or S) link two non-adjacent carbon or nitrogen atoms. Preferred bridges include, but are not limited to, one carbon atom, two carbon atoms, one nitrogen atom, two nitrogen atoms, and a carbon-nitrogen group. It is noted that a bridge always converts a monocyclic ring into a tricyclic ring. When a ring is bridged, the substituents recited for the ring may also be present on the bridge. Spiro and fused rings are also included.

As used herein, the term “aromatic heterocycle” or “heteroaryl” is intended to mean a stable 5, 6, or 7-membered monocyclic or bicyclic aromatic heterocyclic ring or 7, 8, 9, 10, 11, or 12-membered bicyclic aromatic heterocyclic ring which consists of carbon atoms and one or more heteroatoms, e.g., 1 or 1-2 or 1-3 or 1-4 or 1-5 or 1-6 heteroatoms, independently selected from the group consisting of nitrogen, oxygen, and sulfur. In the case of bicyclic heterocyclic aromatic rings, only one of the two rings needs to be aromatic (e.g., 2,3-dihydroindole), though both may be (e.g., quinoline). The second ring can also be fused or bridged as defined above for heterocycles. The nitrogen atom may be substituted or unsubstituted (i.e., N or NR wherein R is H or another substituent, as defined). The nitrogen and sulfur heteroatoms may optionally be oxidized (i.e., N→O and S(O)ₚ, where p=1 or 2). It is to be noted that total number of S and O atoms in the aromatic heterocycle is not more than 1.

Examples of heterocycles include, but are not limited to, acridinyl, azocinyl, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, and xanthenyl.

### Progesterone Receptor Antagonists

The class of drugs known as progesterone receptor antagonists is well known in the art (e.g., Spitz, I M, 2006, Curr. Opin. Investig. Drugs 10:882-90; Wagner et al., 1999, Endocrinology 140:1449-58, U.S. Patent Publication No. 2008/0200440A1, herein incorporated by reference). Examples include Mifepristone, Ulipristal acetate (“Ella”), CDB-2914, CDB-4124 (Proellex, Progenta), ORG-33628 (NV Organon); other examples are described in the references cited and incorporated by reference above. Progesterone receptor antagonist compounds or partial agonists of the progesterone receptor that increase SERCA2a levels in cells are also useful in the methods of reducing heart failure. Asoprisnil (J867) is an example of a partial agonist compound.

### Formulations

Formulations of therapeutically-active compounds include those suitable for oral, sublingual, buccal, parenteral (for example subcutaneous, intramuscular, or intravenous), rectal, topical including transdermal, intranasal and inhalation administration. In one embodiment, mifepristone or its derivative are formulated for oral administration. Regardless of the formulation, the dose of mifepristone is generally in the range from 6 mg/kg to 25 mg/kg. Dose ranges and amounts tailored to individual patients or classes of patients are determined using methods well known in the art.

Formulations suitable for oral administration may be provided as discrete units, such as tablets, capsules, cachets, lozenges, each containing a predetermined amount of mifepristone or its derivative; as powders or granules; as solutions or suspensions in aqueous or non-aqueous liquids; or as oil-in-water or water-in-oil emulsions. In one embodiment, the mifepristone or derivative thereof is manufactured as a slow release formulation, e.g., a dermal patch. In the latter example, the compound is formulated with a penetration enhancer and delivery of the therapeutically-active compound to the subject continues over a period of hours, weeks, and even 1, 2, 3 months (see, e.g., U.S. Patent Publication 20110182972, hereby incorporated by reference) to confer clinical benefit for a prolonged period of time.

Other formulations for sustained-release (SR), sustained-action (SA), extended-release (ER, XR, or XL), time-release or timed-release, controlled-release (CR), modified release (MR), or continuous-release (CR) of the drug is a mechanism used in pill tablets or capsules to dissolve slowly and release a drug over time. The advantages of sustained-release tablets or capsules are that they can often be taken less frequently than instant-release formulations of the same drug, and that they keep steadier levels of the drug in the bloodstream for extended periods of time. For example, the active ingredient is embedded in a matrix of insoluble substance(s) such that the dissolving drug must find its way out through the holes in the matrix. Some drugs are enclosed in polymer-based tablets with a laser-drilled hole on one side and a porous membrane on the other side. Stomach acids push through the porous membrane, thereby pushing the drug out through the laser-drilled hole. Over time, the entire drug dose releases into the system while the polymer container remains intact, to be excreted later through normal digestion.

In one embodiment, the formulation is a capsule, where the encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

Formulations of the invention may be prepared by any suitable method, typically by uniformly and intimately admixing mifepristone or its derivative with liquids or finely divided solid carriers or both, in the required proportions and then, if necessary, shaping the resulting mixture into the desired shape.

For example a tablet may be prepared by compressing an intimate mixture comprising a powder or granules of mifepristone or its derivative and one or more optional ingredients, such as a binder, lubricant, inert diluent, or surface active dispersing agent, or by moulding an intimate mixture of powdered active ingredient and inert liquid diluent.

In addition to the ingredients specifically mentioned above, the oral formulations of the present invention may include other agents known to those skilled in the art of pharmacy, having regard for the type of formulation in issue. Oral formulations suitable may include flavoring agents.

In another embodiment, the formulation comprises about 0.1 mg to about 1500 mg of mifepristone or its derivative per tablet. In another embodiment, the formulation comprises about 1 mg to about 100 mg. In another embodiment, the formulation comprises about 1 mg to about 50 mg. In another embodiment, the formulation comprises about 5 mg to about 25 mg.

All percentages and ratios used herein, unless otherwise indicated, are by weight. The percent dimeric impurity is on an area percent basis, typically as quantified by analytical HPLC.

Throughout the description, where compositions are described as having, including, or comprising specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the invention remains operable. Moreover, two or more steps or actions can be conducted simultaneously.

### Heart Failure and Sudden Cardiac Death

Dysfunctional calcium cycling proteins in cardiomyocytes, including L-type calcium channel (LTCC), ryanodine receptor (RyR-2), SERCA2a, and phospholamban (PLN), are associated with heart failure and cardiac arrhythmia. During the excitation-contraction coupling in cardiomyocytes, LTCC on cell membrane is activated by and calcium entry through this channel triggers calcium release from the sarcoplasmic reticulum (SR) through the RyR-2 receptor/channel located on its membrane of the SR. Cardiac relaxation is an active process mediated by the pumping of calcium back into the SR by an SR pump encoded by SERCA2a. This pump is in turn regulated by phospholamban (PLN). In heart failure in human and experimental models, the steady state level of the protein and activity of SERCA2 is down regulated and the RYR-2 receptor is leaky. This down regulation is associated with increased calcium concentration in the cytosol which leads to abnormal systolic and diastolic function of the heart, and abnormal function of membrane ion channels such as LTCC, which lead to triggered activity (early and late after depolarizations) that initiates cardiac arrhythmias and SCD.

The overexpression of SERCA2a has been established in vitro and in vivo as a method to improve cardiac function in heart failure. Indeed, overexpression of SERCA2a increases the contractility in failing cardiomyocytes and reduces the susceptibility to ventricular arrhythmia in small and large animal models. In clinical trials, adeno-associated virus (AAV) is used as a vector to deliver SERCA2 into the heart (cardiomyocytes) of patients with heart failure via intracoronary injection. Preliminary data show that it can decrease mortality, reduced hospitalization and prevent complication rate in patients with stage four heart failure. However prior to the invention, there were no known small molecules that can increase the expression of SERCA2a.

Long-term administration of RU486 has been evaluated in the treatment of meningioma and demonstrated to be clinically tolerated. The common side effects include fatigue, hot flush and gynecomastia.

### RU486 Increases SERCA2 in Cardiomyocytes

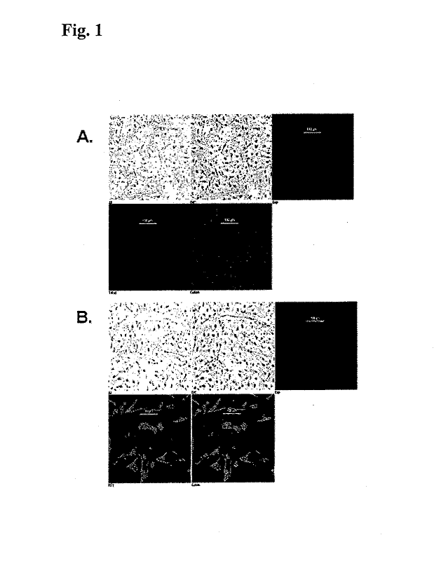

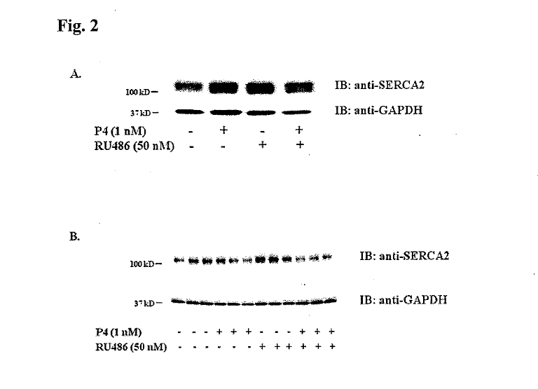

It was a surprising discovery that RU486 increases the expression of SERCA2 polypeptides in cultured neonatal rabbit ventricular cardiomyocytes (NRbVCM) by 60%. Ventricular cardiomyocytes were isolated in neonatal wild type NZW rabbits (3-5 days old, 70-110 g) with enzymatic techniques. The hearts were removed from euthanized rabbits and perfused for 5 to 7 minutes with a free solution containing 140 mM NaCl, 4.4 mM KCl, 1.5 mM MgCl₂, 0.33 mM NaH₂PO₄, 16 mM taurine, 5 mM HEPES, 5 mM pyruvic acid, and 7.5 mM glucose. Subsequently, the hearts were perfused for 10 to 15 minutes with the same solution to which 0.3 mg/ml collagenase was added. The whole ventricles were cut off and minced, and the cells were dispersed in a solution containing 45 mM KCl, 65 mM K-glutamate, 3 mM MgSO₄, 15 mM KH₂PO₄, 16 mM taurine, 10 mM HEPES, 0.5 mM EDTA, and 10 mM glucose (pH 7.3). The cell suspension was filtered through a 100-pm cell strainer and kept at room temperature for one hour. Thereafter, the cells were gradually recovered with MEM, in which Ca²⁺ concentration was increased from 100 μM, 500 μM, 1000 μM and 1800 μM. Every well of six-well plate was seed one million cells, cultured in DMEM (Lonza 12-708F,) with 7% FBS and 100 μM of BrdU. Medium was changed every 24 to 48 hours. Cells were maintained at 37° C. with 5% CO2 (FIG. 1). On day four of culturing, the cells are treated with either 1 nM of progesterone (P4) or 50 nM of RU486 for 48 hours or both (FIGS. 2&3). On day six, the cells are harvested and processed for Western blot per protocol. The SERCA2 is detected by anti-SERCA2 antibody. The level of SERCA2 was normalized against GAPDH.

### Methods of Treatment

The data described herein indicate that RU486-induced increase of SERCA2 in cardiomyocytes presents an effective approach for the therapy of heart failure in a subject in need thereof.

A “subject” in the context of the present invention is preferably a mammal. The mammal can be a human, non-human primate, mouse, rat, dog, cat, horse, or cow, but are not limited to these examples. A subject can be one who has been previously diagnosed or identified as having reduced systolic and/or diastolic function of the heart causing heart failure, and optionally has already undergone, or is undergoing, a therapeutic intervention for the heart failure. Alternatively, a subject can also be one who has not been previously diagnosed as having failure, but who is at risk of developing such condition, e.g. due to coronary artery disease and heart attack, high blood pressure (hypertension), faulty heart valves, damage to the heart muscle (cardiomyopathy), myocarditis, or other diseases, for example chronic diseases such as diabetes, severe anemia, hyperthyroidism, hypothyroidism, emphysema, lupus, hemochromatosis and buildup of proteins in the muscles (amyloidosis). For example, a subject can be one who exhibits one or more symptoms for heart failure. Alternatively, the subject has not been diagnosed with Cushing's Disease.

Low SERCA2a activity correlates with low ejection fraction (systolic heart failure). Accordingly, the composition of the present invention can be administered to a subject with systolic heart failure and ejection fraction less than 40%. Clinically, these are subjects with NYHA class 3 and class 4 heart failure:

Class III: marked limitation of any activity; the patient is comfortable only at rest.

Class IV: any physical activity brings on discomfort and symptoms occur at rest.

Alternative, the composition of the present invention can be administered to a subject with diastolic heart failure and normal ejection fraction which often occurs in hypertensive heart disease (or idiopathic diastolic heart failure). The composition of the present invention may also be administered to patients with ejection fraction less that 30% (with or without symptoms of heart failure for prevention of sudden cardiac death).

A subject can be diagnosed for heart failure by any known methods and/or criteria available in the art, for example, by Framingham criteria. By the Framingham criteria, diagnosis of congestive heart failure (heart failure with impaired pumping capability) requires the simultaneous presence of at least 2 of the following major criteria or 1 major criterion in conjunction with 2 of the following minor criteria:

### Major Criteria:

Cardiomegaly on chest radiography

S3 gallop (a third heart sound)

Acute pulmonary edema

Paroxysmal nocturnal dyspnea

Crackles on lung auscultation

Central venous pressure of more than 16 cm H2O at the right atrium

Jugular vein distension

Positive abdominojugular test

Weight loss of more than 4.5 kg in 5 days in response to treatment (sometimes classified as a minor criterium)

### Minor Criteria:

Tachycardia of more than 120 beats per minute

Nocturnal cough

Dyspnea on ordinary exertion

Pleural effusion

Decrease in vital capacity by one third from maximum recorded

Hepatomegaly

Bilateral ankle edema

Minor criteria are acceptable only if they cannot be attributed to another medical condition such as pulmonary hypertension, chronic lung disease, cirrhosis, ascites, or the nephrotic syndrome. The Framingham Heart Study criteria are 100% sensitive and 78% specific for identifying persons with definite congestive heart failure.

The terms “treating” and “treatment” as used herein refer to the administration of an agent or formulation to a clinically symptomatic individual afflicted with an adverse condition, disorder, or disease, so as to effect a reduction in severity and/or frequency of symptoms, eliminate the symptoms and/or their underlying cause, and/or facilitate improvement or remediation of damage. The terms “preventing” and “prevention” refer to the administration of an agent or composition to a clinically asymptomatic individual who is susceptible or predisposed to a particular adverse condition, disorder, or disease, and thus relates to the prevention of the occurrence of symptoms and/or their underlying cause.

By the terms “effective amount” and “therapeutically effective amount” of a formulation or formulation component is meant a sufficient amount of the formulation or component to provide the desired effect. For example, “an effective amount” of a vaccine is an amount of a compound required to blocking red blood cells (RBCs) rupture. Ultimately, the attending physician or veterinarian decides the appropriate amount and dosage regimen.

The composition of the invention can be administered alone or in combination with a second composition. For example, the primary composition is not a nucleic acid. Preferably, the second composition does not comprise a non-nucleic acid based composition. The composition of the invention can also be administered with one or more diuretic agents (e.g., furosemide or benzthiazide) as preventive treatment post myocardial infarction. The compound of the invention can be administered prior to, concurrently, or after other therapeutic agents such as low dose beta blockers to prevent arrhythmias and/or SCD, ACE inhibitors, or angiotensin receptor blockers.

The composition of the present invention can be administered orally, parenterally, intraperitoneally, intravenously, intraarterially, transdermally, sublingually, intramuscularly, rectally, transbuccally, intranasally, liposomally, via inhalation, vaginally, intraoccularly, via local delivery by catheter or stent, subcutaneously, intraadiposally, intraarticularly, intrathecally, or in a slow release dosage form. Preferably, the antibody is administered intravenously or orally.

The dose of the present composition is measured in units of mg/kg of body weight. The dose can also be measured in units of mg/kg of lean body weight (i.e., body weight minus body fat content). Alternatively, the dose is measured in units of mg/m2 of body surface area. In other embodiments, the dose is measured in units of mg per dose administered to a patient. Any measurement of dose can be used in conjunction with the compositions and methods of the invention and dosage units can be converted by means standard in the art.

The dose for the compound can be between 0.1-50 mg/kg (e.g., 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg). The frequency of administration is preferably once every 7 to 21 days (e.g., once every 7, 10, 14, 18, 21 days). Alternatively, the frequency of administration is preferably 1, 2, or 3 times every 7 to 21 days (e.g., once every 7, 10, 14, 18, 21 days). In some embodiments, 2-20 doses (e.g., 2, 4, 6, 8, 10, 12, 14, 16, 18, 20 doses) are given. The preferred route of administration is oral.

In one embodiment, the formulation is a combination of mifepristone or its derivative and a mineralocorticoid (aldosterone) receptor antagonist. In one embodiment, the amount of mineralocorticoid (aldosterone) receptor antagonist is about 25 mg, about 30 mg, about 35 mg, about 40 mg, about 45 mg, 50 mg, about 55 mg, about 60 mg, about 65 mg, about 70 mg, 75 mg, about 80 mg, about 85 mg, about 90 mg, or about 100 mg. In one embodiment, the amount of mineralocorticoid (aldosterone) receptor antagonist is about 25 mg, about 50 mg, about 75 mg, or 150 mg and up to 200 or 500 mg. In one embodiment, the mineralocorticoid (aldosterone) receptor antagonist is spironolactone (7α-Acetylthio-3-oxo-17α-pregn-4-ene-21,17-carbolactone; marketed under the trade names Aldactone, Novo-Spiroton, Aldactazide, Spiractin, Spirotone, Verospiron or Berlactone). A preferred dose range for spironolactone is 25-100 mg. In another embodiment, the mineralocorticoid (aldosterone) receptor antagonist is eplerenone (pregn-4-ene-7,21-dicarboxylic acid, 9,11-epoxy-17-hydroxy-3-oxo, γ-lactone, methyl ester (7α, 11α, 17α)). In one embodiment, the amount of eplerenone is about 25 mg, about 30 mg, about 35 mg, about 40 mg, about 45 mg, or about 75 mg. In another embodiment, the amount of eplerenone is about 25 mg. A preferred dose range for eplerenone is 25-50 mg.

Two drugs are optionally formulated in the same pill by (a) adding a mineralocorticoid (aldosterone) receptor antagonist such as spironolactone (25, 50, 75, or 100 mg to mifepristone or (b) adding eplerenone (25 mg) to mifepristone. Spironolactone and eplerenone are commercially available, e.g., from Pfizer. Both drugs function as potassium sparing diuretics.

The dosing regimen that can be used in the methods of the invention includes, but is not limited to, daily, three times weekly (intermittent), two times weekly, weekly, or every 14 days. Alternatively, dosing regimen includes, but is not limited to, monthly dosing or dosing every 6-8 weeks. The compound of the present invention can be administered orally once, twice, three times or more alone or in combination with 1, 2, 3, 4, or more additional therapeutic agents in a subject, preferably a human subject. The additional therapeutic agent is a non-nucleic acid based composition or a diuretic agent. Examples of diuretic agent includes, not is not limited to, amphotericin B, lithium citrate, Goldenrod, Juniper, dopamine, acetazolamide, dorzolamide, bumetanide, ethacrynic acid, furosemide, torsemide, glucose (especially in uncontrolled diabetes), mannitol, amiloride, spironolactone, triamterene, potassium canrenoate, bendroflumethiazide, hydrochlorothiazide, caffeine, theophylline, theobromine.

“Measuring” or “measurement,” or alternatively “detecting” or “detection,” or “assessing” or “assessment” means assessing the presence, absence, quantity or amount (which can be an effective amount) of either a given substance within a clinical or subject-derived sample, including the derivation of qualitative or quantitative concentration levels of such substances, or otherwise evaluating the values or categorization of a subject's non-analyte clinical parameters.

The actual measurement of levels or amounts of SERCA2a can be determined at the protein or nucleic acid level using any method known in the art. For example, at the nucleic acid level, Northern and Southern hybridization analysis, as well as ribonuclease protection assays using probes which specifically recognize one or more of these sequences can be used to determine gene expression. Alternatively, amounts of SERCA2a can be measured using reverse-transcription-based PCR assays (RT-PCR), e.g., using primers specific for the differentially expressed sequence of genes or by branch-chain RNA amplification and detection methods by Panomics, Inc. Amounts of SERCA2a can also be determined at the protein level, e.g., by measuring the levels of peptides encoded by the gene products described herein, or subcellular localization or activities thereof using technological platform such as for example AQUA® (HistoRx, New Haven, Conn.) or U.S. Pat. No. 7,219,016. Such methods are well known in the art and include, e.g., immunoassays based on antibodies to proteins encoded by the genes, aptamers or molecular imprints. Any biological material can be used for the detection/quantification of the protein or its activity. Alternatively, a suitable method can be selected to determine the activity of proteins encoded by the marker genes according to the activity of each protein analyzed. In this method, a biological sample can be provided from a subject undergoing treatment regimens, e.g., drug treatments, for heart failure. If desired, biological samples are obtained from the subject at various time points before, during, or after treatment. A “sample” can be any bodily fluid or tissue sample obtained from a subject, including, but is not limited to, blood, blood serum, urine, and saliva.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. Genbank and NCBI submissions indicated by accession number cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

1. A composition for reducing heart failure in a subject in need thereof, comprising a compound that increases cellular level of Sarco/endoplasmic reticulum Ca²⁺-ATPase 2a (SERCA2a).

2. The composition of claim 1, wherein said composition comprises mifepristone or a derivative thereof.

3. The composition of claim 1, wherein said composition further comprises a progesterone receptor modulator, said modulator comprising an antagonist or partial agonist.

3. A method of reducing heart failure in a subject in need thereof, comprising administering a composition comprising a composition that increases cellular level of Sarco/endoplasmic reticulum Ca²⁺-ATPase 2a (SERCA2a).

4. The method of claim 3, wherein said composition comprises mifepristone or a derivative thereof.

5. The method of claim 3, wherein said composition comprises a progesterone receptor modulator, said modulator comprising an antagonist or partial agonist.

6. The method of claim 3, wherein said heart failure comprises systolic and/or diastolic heart failure.

7. The method of claim 3, wherein said composition further comprises a potassium sparing diuretic.

8. The method of claim 7, wherein said potassium sparing diuretic comprises spironolactone or eplerenone.

9. The method of claim 3, wherein said subject has not been diagnosed with Cushing's Disease.

10. The method of claim 4, further comprising administering a second composition comprising a non-nucleic acid based composition.

11. A method of preventing or inhibiting a cardiovascular disease in a subject in need thereof, comprising
a) assessing the level of cardiac impairment or determining the level of Sarco/endoplasmic reticulum Ca²⁺-ATPase 2a (SERCA2a) in said subject;
b) administering to said subject a therapeutically effective amount of mifepristone, in an amount effective to increase the level of SERCA2a;
c) re-assessing the level of cardiac impairment and the level of SERCA2a in said subject; whereby a reduction in the level of cardiac impairment and an increase in the level of SERCA2a after administration of mifepristone is indicative of reduced risk of having a cardiovascular disease in said subject.

12. The method of claim 3, wherein said subject is characterized by a level of cardiac impairment comprising class III or class IV heart failure or diastolic heart failure.

13. A composition comprising mifepristone in a slow release composition.

14. The composition of claim 13, wherein said composition is formulated as a dermal patch.

15. A composition comprising mifepristone and a second agent comprising a potassium sparing diuretic.

16. The composition of claim 15, wherein said potassium sparing diuretic is selected from the group consisting of spironolactone and eplerenone.
